(19) United States
(12) Patent Application Publication
(10) Pub. No.: US 2023/0271958 A1
(43) Pub. Date: Aug. 31, 2023

(54) SALT AND CRYSTAL FORM OF DIHYDROPYRIDO[2,3-d]PYRIMIDINE DERIVATE

(71) Applicant: NANJING CHIA TAI TIANQING PHARMACEUTICAL CO., LTD., Nanjing (CN)

(72) Inventors: Jian WU, Nanjing (CN); Changyou MA, Nanjing (CN); He TIAN, Nanjing (CN); Jianliang ZHAO, Nanjing (CN); Donghui CHEN, Nanjing (CN); Dan XU, Nanjing (CN); Chunxia ZHU, Nanjing (CN); Zhoushan TIAN, Nanjing (CN)

(73) Assignee: NANJING CHIA TAI TIANQING PHARMACEUTICAL CO., LTD., Nanjing (CN)

(21) Appl. No.: 18/017,421

(22) PCT Filed: Jul. 22, 2021
(86) PCT No.: PCT/CN2021/107814

§ 371 Date: Jan. 23, 2023

(30) Foreign Application Priority Data
Jul. 22, 2020 (CN) 202010709837.9

(51) Int. Cl. C07D 471/04; A61P 35/00

(52) U.S. Cl. CPC C07D 471/04; A61P 35/00; C07B 2200/13

(57) ABSTRACT

A salt and crystal form of a dihydropyrido[2,3-d]pyrimidine derivate, and specifically, a crystal form of a fumarate hydrate of compound 1, and a preparation method thereof are provided. In the formula, X is 2.0-3.0. The X-ray powder diffraction pattern expressed in 2θ angles using Cu-Ka radiation has characteristic peaks at 2θ values of 9.28°±0.2° and 3.63°±0.2°. The crystal form has good stability and can better be applied to clinical practice.

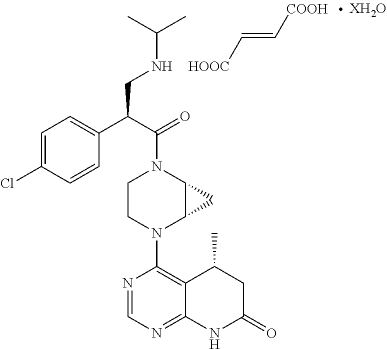 [SMILES: CC(C)NC[C@@H](C(=O)N1CCN(c2ncnc3c2[C@H](C)CC(=O)N3)[C@H]2C[C@H]21)c1ccc(Cl)cc1 ; O=C(O)/C=C/C(=O)O]

The present application claims priority to Chinese Patent Application No. 202010709837.9, entitled “Salt and Crystal Form of Dihydropyrido[2,3-d]pyrimidine Derivate” and filed with the China Patent Office on Jul. 22, 2020, the entire content of which is incorporated herein by reference.

### TECHNICAL FIELD

The present application belongs to the field of medicinal chemistry, and specifically relates to a salt of dihydropyrido[2,3-d]pyrimidinone derivative, a crystal form thereof, and a preparation method and medical use thereof.

### BACKGROUND

The PI3K/AKT/mTOR pathway consisting of phosphoinositide-3-kinase (PI3K) and its downstream protein AKT (also known as protein kinase B, PKB), and mammalian target of Rapamycin (mTOR) as a very important intracellular signal transduction pathway, the pathway exerts an extremely important biological function in the process of cell growth, survival, proliferation, apoptosis, angiogenesis, autophagy, etc. Abnormal activation of the pathway will cause a series of diseases such as cancer, neuropathy, autoimmune disease, and hemolymphatic system disease.

AKT is a type of serine/threonine kinase and affects the survival, growth, metabolism, proliferation, migration, and differentiation of cell through numerous downstream effectors. Overactivation of AKT has been observed in more than 50% of human tumors, especially in prostate cancer, pancreatic cancer, bladder cancer, ovarian cancer, and breast cancer. Overactivation of AKT may lead to the formation, metastasis, and drug resistance of tumor.

AKT has three isoforms: AKT1, AKT2, and AKT3. As a typical protein kinase, each isoform consists of an amino-terminal pleckstrin homology (PH) domain, a middle ATP-binding kinase domain, and a carboxyl-terminal regulatory domain. About 80% amino acid sequences of the three isoforms are homologous, and only the amino acid sequences in a binding domain between the PH domain and the kinase domain changes greatly.

The current drugs targeting the PI3K/AKT/mTOR signaling pathway mainly include PI3K inhibitors and mTOR inhibitors, while AKT is at the core of the signal transduction pathway. Inhibition of the AKT activity can not only avoid the severe side effects caused by inhibition of upstream PI3K, but also avoid the negative feedback mechanism caused by inhibition of downstream mTOR from affecting the efficacy of a drug. For example, CN101631778A discloses a class of cyclopentadiene[D]pyrimidine derivatives, CN101578273A discloses a class of hydroxylated and methoxylated cyclopentadiene[D]pyrimidine derivatives, CN101511842A discloses a class of dihydrofuropyrimidine derivatives, CN101970415A discloses a class of 5H-cyclopentadiene[d]pyrimidine derivatives, and these compounds inhibit AKT1 with IC₅₀ less than 10 μM. However, development of effective and selective AKT inhibitors is still an important direction for current development of tumor-targeting drugs.

### SUMMARY OF THE INVENTION

In one aspect, the present application provides a crystal form (hereinafter referred to as crystal form A) of a fumarate hydrate having the following structure:

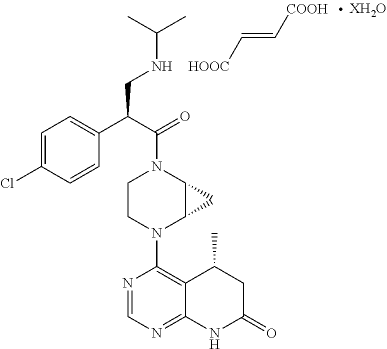

where, X is 2.0-3.0, and
an X-ray powder diffraction pattern expressed in 2θ angles using Cu-Ka radiation has characteristic peaks at 20 values of 9.28°±0.2° and 3.63°±0.2°.

The above said fumarate hydrate is a fumarate hydrate of compound 1, wherein the compound 1 has the following structure:

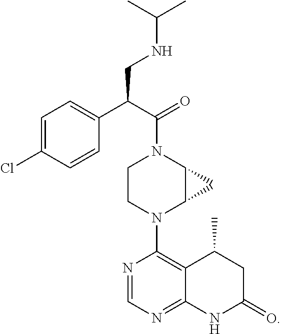 [SMILES: CC(C)NC[C@@H](C(=O)N1CCN(c2ncnc3c2[C@H](C)CC(=O)N3)[C@H]2C[C@H]21)c1ccc(Cl)cc1]

In some embodiments, the X-ray powder diffraction pattern expressed in 20 angles of crystal form A has characteristic peaks at 20 values of 9.28°±0.2°, 19.45°±0.2°, 21.60°±0.2°, and 23.63°±0.2°.

In some embodiments, the X-ray powder diffraction pattern expressed in 20 angles of crystal form A has characteristic peaks at 20 values of 9.28°±0.2°, 14.22°±0.2°, 19.45°±0.2°, 21.60°±0.2°, and 23.63°±0.2°.

In some embodiments, the X-ray powder diffraction pattern expressed in 20 angles of crystal form A has characteristic peaks at 20 values of 9.28°±0.2°, 10.72°±0.2°, 14.22°±0.2°, 19.45°±0.2°, 21.60°±0.2°, 23.63°±0.2°, 24.50°±0.2°, 24.83°±0.2°, 25.08°±0.2°, and 30.33°±0.2°.

In some embodiments, the X-ray powder diffraction pattern expressed in 20 angles of crystal form A has characteristic peaks at 20 values of 5.29°±0.2°, 9.28°±0.2°, 10.72°±0.2°, 11.24°±0.2°, 12.13°±0.2°, 12.51°±0.2°, 13.60°±0.2°, 14.22°±0.2°, 15.64±0.2°, 16.14°±0.2°, 16.52°±0.2°, 17.38°±0.2°, 17.99°±0.2°, 18.68°±0.2°, 19.00°±0.2° 19.45°±0.2° 19.80°±0.2° 20.53°±0.2° 21.60°±0.2° 21.89°±0.2° 22.58°±0.2° 23.63°±0.2°, 24.50°±0.2° 24.83°±0.2° 25.08°±0.2° 25.66°±0.2° 26.09°±0.2° 26.84°±0.2° 27.43°±0.2° 27.94°±0.2°, 28.81°±0.2° 29.52°±0.2° 29.98°±0.2° 30.33°±0.2° 30.92°±0.2° 32.03°±0.2° 32.80°±0.2° 33.34°±0.2°, 34.14°±0.2°, 34.72°±0.2°, 35.83°±0.2°, 36.55°±0.2°, 37.35°±0.2°, 38.11°±0.2°, and 38.93°±0.2°.

In some embodiments, the X-ray powder diffraction pattern expressed in 20 angles of crystal form A is shown as FIG. 4.

In some embodiments, the X-ray powder diffraction pattern expressed in 20 angles of crystal form A is shown as FIG. 8.

In some embodiments, the X-ray powder diffraction pattern expressed in 20 angles of crystal form A is shown as FIG. 10.

In some embodiments, a thermogram of crystal form A that is obtained by differential scanning calorimetry (DSC) has an endothermic peak at the onset temperature of 118-128° C.

In some embodiments, the thermogram of crystal form A that is obtained by DSC has an endothermic peak at the onset temperature of 120-125° C.

In some embodiments, the thermogram of crystal form A that is obtained by DSC has an endothermic peak at the onset temperature of 123° C.

In some typical embodiments, the DSC pattern of crystal form A is shown as FIG. 5.

In some embodiments, a spectrum of crystal form A that is obtained by attenuated total reflectance Fourier transform infrared spectroscopy has the following absorption bands expressed in reciprocals of wavelengths (cm-1): 3451±2, 2981±2, 2953±2, 2882±2, 2824±2, 2477±2, 1698±2, 1631±2, 1596±2, 1544±2, 1490±2, 1465±2, 1441±2, 1390±2, 1362±2, 1320±2, 1302±2, 1283±2, 1254±2, 1197±2, 1135±2, 1091±2, 1058±2, 1014±2, 983±2, 929±2, 894±2, 867±2, 834±2, 802±2, 784±2, 761±2, 739±2, 718±2, 663±2, 647±2, 640±2, 584±2, 560±2, and 497±2. In some embodiments, a spectrum of crystal form A that is obtained by Fourier transform Raman spectroscopy has the following absorption bands expressed in reciprocals of wavelengths (cm-1): 1699±2, 1664±2, 1602±2, 1340±2, 867±2, 829±2, 809±2, 747±2, and 669±2.

In some embodiments, the thermogravimetric analysis (TGA) pattern of crystal form A is shown as FIG. 6.

In some embodiments, the TGA pattern of crystal form A is shown as FIG. 7.

In some embodiments, the TGA pattern of crystal form A is shown as FIG. 9.

In some typical embodiments, crystal form A is a hydrate containing 2.0-2.5 water molecules, that is, X in the structural formula is 2.0-2.5.

In another aspect, the present application provides a crystal form composition of crystal form A, the weight of crystal form A accounts for more than 50% of the weight of the crystal form composition, preferably more than 80%, further preferably more than 90%, much further preferably more than 95%, and the most preferably more than 98%.

In another aspect, the present application also provides a pharmaceutical composition comprising crystal form A or the crystal form composition.

In some embodiments, the pharmaceutical composition further includes one or more pharmaceutically acceptable carriers.

In some embodiments, the pharmaceutical composition is a solid pharmaceutical preparation suitable for oral administration, and preferably tablets or capsules.

In another aspect, the present application also provides crystal form A or a crystal form composition or a pharmaceutical composition that is used as a medicament.

In another aspect, the present application also provides use of crystal form A or a pharmaceutical composition thereof in the preparation of a medicament for preventing and/or treating an AKT protein kinase-mediated disease or disease state.

In another aspect, the present application also provides use of the crystal form composition in the preparation of a medicament for preventing and/or treating an AKT protein kinase-mediated disease or disease state.

In another aspect, the present application also provides use of crystal form A or a pharmaceutical composition thereof in the prevention and/or treatment of an AKT protein kinase-mediated disease or disease state.

In another aspect, the present application also provides use of the crystal form composition in the prevention and/or treatment of an AKT protein kinase-mediated disease or disease state.

In another aspect, the present application also provides a method for preventing and/or treating an AKT protein kinase-mediated disease or disease state, which includes a step of administering crystal form A or a pharmaceutical composition thereof of the present application to the subject in need.

In another aspect, the present application also provides a method for preventing and/or treating an AKT protein kinase-mediated disease or disease state, which includes a step of administering the crystal form composition of the present application to the subject in need.

In another aspect, the present application also provides crystal form A or a pharmaceutical composition thereof of the present application that is used for preventing and/or treating an AKT protein kinase-mediated disease or disease state.

In another aspect, the present application also provides the crystal form composition of the present application that is used for preventing and/or treating an AKT protein kinase-mediated disease or disease state.

In some embodiments, the AKT protein kinase-mediated disease or disease state is cancer.

In some typical embodiments, the cancer is breast cancer, prostate cancer or ovarian cancer.

In some typical embodiments, the cancer is prostate cancer.

Relevant Definitions

Unless otherwise specified, the following terms used in the description and claims have the following meanings.

The term “pharmaceutically acceptable carrier” refers to a carrier that has no obvious stimulating effect on the body and will not impair the biological activity and performance of an active compound. Pharmaceutically acceptable carriers include, but are not limited to, any diluent, disintegrant, adhesive, glidant, and wetting agent that have been approved by the National Medical Products Administration for human or animal use.

The “X-ray powder diffraction pattern” in the present application is obtained by using Cu-Ka radiation.

“2θ” or “2θ” in the present application refers to a diffraction angle, θ is a Bragg angle in ° or degrees, and an error range of each characteristic peak 2θ is ±0.2θ°.

It should be noted that a diffraction pattern of a crystal compound that is obtained by X-ray powder diffraction (XRPD) spectrum is often characteristic for particular crystals, and in the pattern, relative intensities of bands (especially at low angles) may vary due to a preferred orientation effect caused by differences in crystallization conditions, particle size, and other measurement conditions. Therefore, relative intensities of diffraction peaks are not characteristic for the targeted crystals. When judging whether it is identical to a known crystal, more attention should be paid to relative positions of peaks rather than their intensities. In addition, it is also well known in the field of crystallography that for any given crystal, there may be slight errors in positions of peaks. For example, due to changes of temperature, movements of a sample or calibration of an instrument during analysis of the sample, positions of peaks may be moved, and a measurement error of a 2θ value is sometimes about ±0.2°. Therefore, the error should be taken into account when a crystal structure is determined. In an XRPD pattern, a 2θ angle or interplanar spacing d is usually used to indicate the position of a peak, and there is a simple conversion relationship between the two: d=)λ2 sinθ, where, d is interplanar spacing, is the wavelength of an incident X-ray, and θ is a diffraction angle. For the same crystal of the same compound, positions of peaks in its XRPD pattern are similar on the whole, while a relative intensity error may be large. It should also be noted that in identification of a mixture, some diffracted rays will be lost due to factors such as content decline. In this case, there is no need to rely on all bands observed in a high-purity sample, even a single band may be characteristic for the given crystal.

Differential scanning calorimetry (DSC) is a technique for determining the transition temperature at which a crystal absorbs or releases heat due to a change in its crystal structure or melting of the crystal. For the same crystal form of the same compound, in continuous analysis, errors of the thermal transition temperature and a melting point are typically within about 5° C., and usually within about 3° C. When it is described that a compound has a given DSC peak or melting point, it is meant that the DSC peak or melting point has an error of ±5° C. DSC is an auxiliary method for distinguishing different crystal forms. Different crystal forms can be identified according to their different transition temperature characteristics. It should be noted that for a mixture, its DSC peak and melting point will fluctuate in a larger range. In addition, the melting of a substance is accompanied by decomposition, so the melting temperature is related to a heating rate.

Thermogravimetric analysis (TGA) is a thermal analysis technique for determining a relationship between the mass of a sample to be tested and changes of temperature at programmed temperature. If a substance to be tested undergoes sublimation or vaporization during heating and the gas is decomposed or the crystal water is lost, which will cause the mass change of the substance. In this case, a thermogravimetric curve is not a straight line but has a drop. By analyzing the thermogravimetric curve, the temperature at which the substance to be tested changes can be known, and how much mass is lost can be calculated according to the lost weight.

When referring, for example, an XRPD pattern, a DSC pattern or a TGA pattern, the term “as shown in . . . ” includes patterns that are not necessarily identical to those depicted herein, but fall within the limits of experimental error when considered by those skilled in the art.

Unless otherwise specified, the abbreviations in the present application have the following meanings:
M: mol/L
mM: mmol/L
nM: nmol/L
Boc: tert-butoxycarbonyl
DCM: dichloromethane
DEA: diethylamine
DIEA: N,N-diisopropylethylamine
HATU: 2-(7-azabenzotriazol-1)-N,N,N′,N′-tetramethyluronium hexafluorophosphate
RT: retention time
SFC: supercritical fluid chromatography
h: hour
min: minute
TK: tyrosine kinase
SEB: fluorescent signal enhancer
HTRF: homogeneous time resolved fluorescence
DTT: dithiothreitol

### BRIEF DESCRIPTION OF THE DRAWINGS

In order to more clearly describe the technical solutions of the examples of the present application and the prior art, the drawings that need to be used in the examples and the prior art will be briefly introduced below. Obviously, the drawings in the following description are some embodiments of the present application only, and those skilled in the art may also obtain other drawings according to these drawings.

FIG. 1 is a schematic diagram of a single molecule of compound 1 of Example 1;

FIG. 2 is a schematic diagram of asymmetric structural unit of an oxalate single crystal of compound 1 of Example 1;

FIG. 3 is an XRPD pattern of an amorphous fumarate prepared by method A of Example 2;

FIG. 4 is an XRPD pattern of crystal form A prepared by method B of Example 2;

FIG. 5 is a DSC pattern of crystal form A prepared by method B of Example 2;

FIG. 6 is a TGA pattern of crystal form A prepared by method B of Example 2;

FIG. 7 is a TGA pattern of crystal form A prepared by method A of Example 2;

FIG. 8 is an XRPD pattern of crystal form A prepared by method A of Example 2;

FIG. 9 is a TGA pattern of crystal A of Example 3; and

FIG. 10 is an XRPD pattern of crystal form A of Example 3.

### DETAILED DESCRIPTION OF THE INVENTION

The present application will be described in more detail below with reference to examples below. However, these specific descriptions are for the purpose of describing the technical solutions of the present application only, and are not intended to limit the present application in any manner.

Test conditions of instruments are as follows:
(1) X-ray powder diffractometer (X-ray Powder Diffraction, XRPD)
Instrument model: Bruker D2 Phaser 2^{nd}
X-ray: Cu-Kα, and λ=1.5406
Slit system: emitted slit=0.4°, and received slit=0.075 mm
X-ray light tube setting: tube voltage=30 KV, and tube current=10 mA
Scanning mode: continuous scanning, scan step (° 2θ)=0.043°, and scan range (° 2θ)=3-40°
   (2) Thermogravimetric analyzer (Thermogravimetric, TGA)
Instrument model: TA Instruments TGA55
Purge gas: nitrogen gas
Heating rate: 10° C./min
Heating range: room temperature −300° C.
   (3) Differential scanning calorimeter (Differential Scanning calorimeter, DSC)
Instrument model: TA Instruments DSC25
Purge gas: nitrogen gas
Heating rate: 10° C./min
Heating range: 20-250° C.
   (4) Fourier transform infrared spectrometer (FT-IR)
Instrument model: Thermo Fourier infrared spectrometer IS5
Instrument calibration: polystyrene film
Test condition: KBr pellet method
   (5) Fourier transform Raman spectrometer (FT-Raman)
Instrument model: Nicolet Fourier transform Raman spectrometer DXR780
Exposure time: 20 s
Impressions: 10
Background impressions: 512
Light source: 780 nm
Slit: 400 lines/mm
Laser intensity: 14 mW
Scan range: 50 cm⁻¹-3000 cm⁻¹

### Example 1 Preparation of Compound 1

### Preparation Example 1 Preparation of (R)-4-chloro-5-methyl-5,8-dihydropyrido[2,3-d]pyrimidin-7(6H)-one (intermediate)

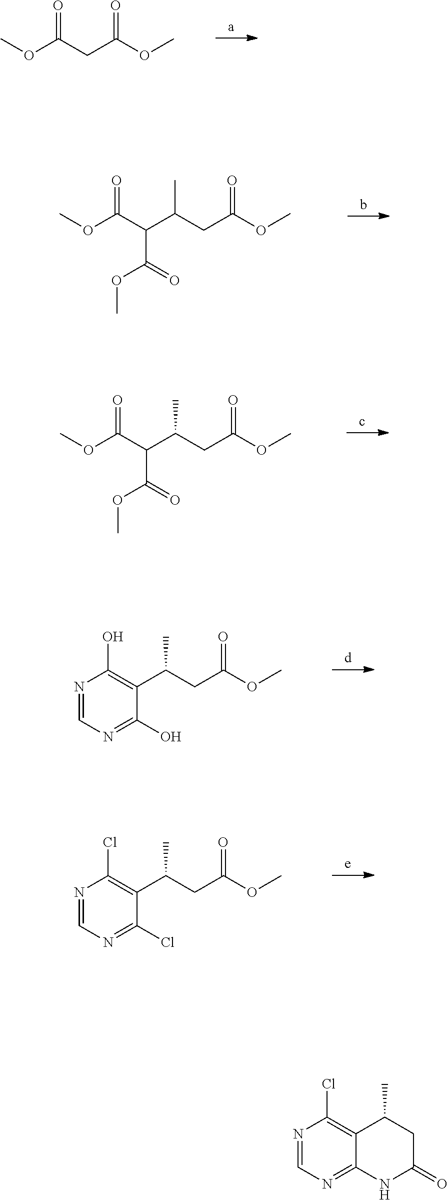

a) Trimethyl 2-methylpropane-1,1,3-tricarboxylate

Under the protection of nitrogen gas, a sodium methylate-methanol solution (30 wt %, 50.32 g) was added to methanol (900 mL), the mixture was heated to 70° C., dimethyl malonate (461.12 g) and ethyl crotonate (349.46 g) were mixed until uniform and dropwise added to the above sodium methylate-methanol solution, and the reaction solution reacted at 70° C. for 3 h. After the reaction was completed, the reaction solution was evaporated under reduced pressure to remove the solvent, ethyl acetate (1 L) was added, the mixture was regulated with 4 M hydrochloric acid until the pH of the mixture was 7-8, water (500 mL) was added, and the solution was separated and evaporated under reduced pressure to remove the organic phase so as to yield a yellow liquid (777.68 g). NMR (400 MHz, DMSO-d₆) δ (ppm) 3.67 (s, 3H), 3.65 (s, 3H), 3.59 (s, 3H), 3.56 (d, J=6.8 Hz, 1H), 2.45-2.58 (m, 2H), 2.23-2.29 (m, 1H), 0.93 (d, J=6.8 Hz, 3H).

b) Trimethyl (R)-2-methylpropane-1,1,3-tricarboxylate

Disodium hydrogen phosphate (4.5 g) was dissolved in deionized water (1.5 L) at 25° C., the solution was regulated with 2 N hydrochloric acid until the pH of the solution was 7.05, trimethyl 2-methylpropane-1,1,3-tricarboxylate (150.46 g) and lipase (Candida rugosa, 40 g, added in 6 d) were added, the mixture was regulated with a 2 N sodium hydroxide solution until the pH of the mixture was 7.0-7.6, and the reaction solution reacted at 35° C. for 6 d. Chirality detection ee %>98%, and chirality detection conditions: Chiralpak IC, 4.6×250 mm, 5 μm, and n-hexane: ethanol=9:1 (volume ratio). The reaction solution was cooled to 10° C. and regulated with 3 M hydrochloric acid until the pH of the reaction solution was 3-4, ethyl acetate (500 mL) was added, the mixture was subjected to suction filtration, an obtained filter cake was washed with ethyl acetate (600 mL), the solution was separated, a saturated sodium bicarbonate aqueous solution (100 mL) was added for washing, the solution was separated, and an obtained organic phase was concentrated to yield a pale-yellow liquid (26.89 g). 1H NMR (400 MHz, CDCl₃) δ (ppm) 3.74 (s, 6H), 3.68 (s, 3H), 3.46 (d, J=7.2 Hz, 1H), 2.71-2.79 (m, 1H), 2.54 (dd, J=15.6, 4.8 Hz, 1H), 2.32 (dd, J=16.0, 8.4 Hz, 1H), 1.06 (d, J=6.8 Hz, 3H).

c) Methyl (R)-3-(4,6-dihydroxypyrimidin-5-yl)butanoate

Under the protection of nitrogen gas, formamidine acetate (11.33 g) was dissolved in methanol (200 mL) at 20° C., the solution was cooled to 0° C., a sodium methylate-methanol solution (30 wt %, 55.62 g) was dropwise added, the reaction solution reacted at 0° C. for 60 min, a methanol (60 mL) solution of trimethyl (R)-2-methylpropane-1,1,3-tricarboxylate (24.07 g) was dropwise added, and the reaction solution was naturally heated to 20° C. and reacted for 10 h. After the reaction was completed, the reaction solution was cooled to 0° C., regulated with 3 N hydrochloric acid until the pH of the reaction solution was 5-6, evaporated under reduced pressure to remove the solvent, cooled to 0° C., and regulated with 3 N hydrochloric acid until the pH of the reaction solution was 3, after a solid was precipitated, the reaction solution was subjected to suction filtration to collect the solid, and an obtained filter cake was washed with ice water (100 mL) and dried in vacuum to yield a white solid (18.79 g) that was directly used at the next step.

d) Methyl (R)-3-(4,6-dichloropyrimidin-5-yl)butanoate

Under the protection of nitrogen gas, methyl (R)-3-(4,6-dihydroxypyrimidin-5-yl)butanoate (14.63 g) was dispersed into acetonitrile (70 mL) at 22° C., phosphorus oxychloride (26.42 g) and diisopropylethylamine (12.51 g) were dropwise added in sequence, the system released heat obviously and was heated to 60° C., the solids were gradually fully dissolved, and the reaction solution reacted for 18 h. After the reaction was completed, the reaction solution was cooled to 0° C., ethyl acetate (100 mL) was added, the mixture was regulated with a saturated sodium bicarbonate solution until the pH of the mixture was 7-8, extracted with ethyl acetate (50 mL×3), and evaporated under reduced pressure to remove the organic phase so as to yield a yellow solid (13.89 g) that was directly used at the next step.

e) (R)-4-chloro-5-methyl-5,8-dihydropyrido[2,3-d]pyrimidin-7(6H)-one

Methyl (R)-3-(4,6-dichloropyrimidin-5-yl)butanoate (13.89 g) and ammonia water (25-28 wt %, 70 mL) were placed in a 100 mL high-pressure kettle at 20° C., and the reaction solution was heated to 50° C. and reacted for 18 h. After the reaction was completed, the reaction solution was cooled to 0° C. and subjected to suction filtration, and an obtained filter cake was beaten with a mixture (30 mL) of petroleum ether and ethyl acetate in a volume ratio of 10:1 to yield a pale-yellow solid (7.32 g). LC-MS (ESI) m/z: 198 (M+H). ¹H NMR (300 MHz, CDCl₃) δ (ppm) 1.30 (d, J=7.2 Hz, 3H), 2.65-2.69 (m, 1H), 2.86-2.92 (m, 1H), 3.47-3.54 (m, 1H), 8.64 (s, 1H), 10.10 (s, 1H).

### Preparation Example 2 Preparation of (R)-4-((1 S,6R)-5-((S)-2-(4-chlorophenyl)-3-(isopropylamino)propionyl)-2,5-diazabicyclo [4.1.0]heptan-2-yl)-5-met hyl-5,8-dihydropyrido[2,3-d]-pyrimidin-7(6H-one (compound 11

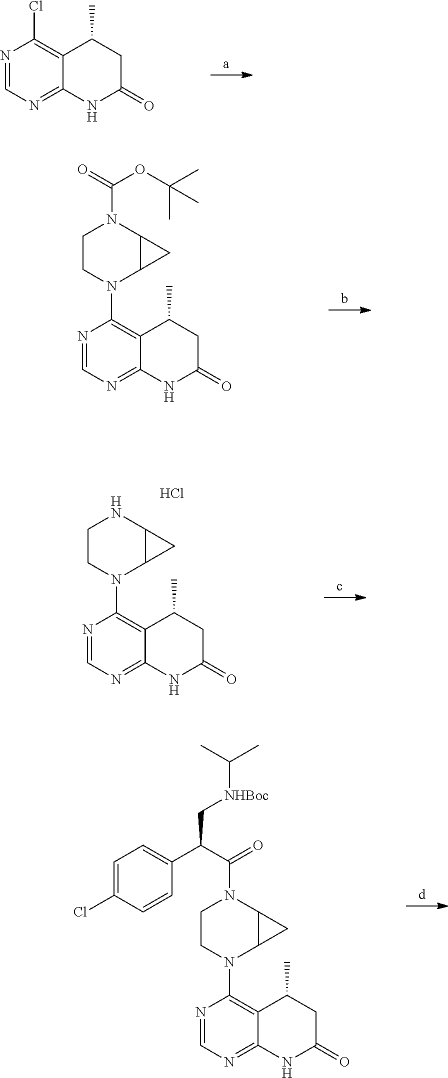

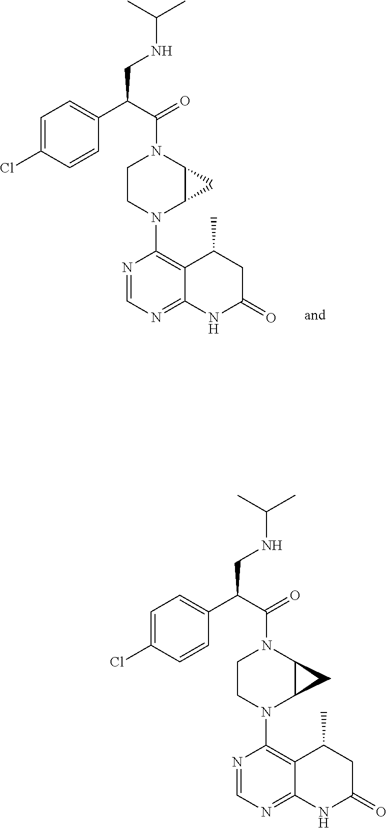 [SMILES: CC(C)NC[C@@H](C(=O)N1CCN(c2ncnc3c2[C@H](C)CC(=O)N3)[C@H]2C[C@H]21)c1ccc(Cl)cc1 ; CC(C)NC[C@@H](C(=O)N1CCN(c2ncnc3c2[C@H](C)CC(=O)N3)[C@@H]2C[C@@H]21)c1ccc(Cl)cc1]

Reaction conditions: a) tert-butyl 2,5-diazabicyclo [4.1.0]heptane-2-carboxylate, N-methylpyrrolidone, and 4-dimethylaminopyridine; b) hydrogen chloride/1,4-dioxane (4.0 M), and dichloromethane; c) (S)-3-((tert-butoxycarbonyl)(isopropyl)amino)-2-(4-chlorophenyl)-propionic acid, 2-(7-benzotriazole oxide)-N,N,N′,N′-tetramethyluronium hexafluorophosphate, 4-dimethylaminopyridine, and N,N-dimethylformamide; and d) trifluoroacetic acid and dichloromethane.
a) Tert-butyl 5-((R)-5-methyl-7-oxo-5,6,7,8-tetrahydropyrido[2,3-d]pyrimidin-4-yl)-2,5-diazabicyclo [4.1.0]heptane-2-carboxylate

Under the protection of nitrogen gas, (R)-4-chloro-5-methyl-5,8-dihydropyrido[2,3-d]pyrimidin-7(6H)-one (0.21 g), tert-butyl 2,5-diazabicyclo[4.1.0]heptane-2-carboxylate (0.31 g), and 4-dimethylaminopyridine (0.39 g) were dissolved in N-methylpyrrolidone (5 mL) at 22° C., and the reaction solution was heated to 140° C. and reacted for 3 h. After the reaction was completed, the reaction solution was cooled to 20° C., poured into ice water (20 mL), extracted with ethyl acetate (20 mL×2), washed with a saturated salt solution (10 mL×3), evaporated under reduced pressure to remove the solvent, and separated by silica gel column chromatography (petroleum ether: ethyl acetate=(3:1)-(1:1)) to yield a pale-yellow liquid (0.28 g). LC-MS (ESI) m/z: 360 (M+H).

b) (5R)-4-(2,5-diazabicyclo [4.1.0]heptan-2-yl)-5-methyl-5,8-dihydropyrido[2,3-d]pyrimidin-7(6H)-one hydrochloride Tert-butyl 5-((R)-5-methyl-7-oxo-5,6,7,8-tetrahydropyrido[2,3-d]pyrimidin-4-yl)-2,5-diazabicyclo [4.1.0]heptane-2-carboxylat e (0.28 g) was dissolved in dichloromethane (5 mL) at 20° C., hydrogen chloride/1,4-dioxane (4.0 mL) was added, and the reaction solution reacted for 1 h. After the reaction was completed, the reaction solution was evaporated under reduced pressure to remove the solvent so as to yield a yellow solid (0.23 g) that was directly used at the next step.
c) Tert-butyl (2S)-2-(4-chlorophenyl)-3-(5-((R)-5-methyl-7-oxo-5,6,7,8-tetrahydropyrido[2,3-d]pyrimidin-4-yl)-2,5-diazabicyclo [4.1.0]heptan-2-yl)-3-oxopropyl)(isopropyl)carbamate

Under the protection of nitrogen gas, (5R)-4-(2,5-diazabicyclo[4.1.0]heptan-2-yl)-5-methyl-5,8-dihydropyridin [2,3-d]pyrimidin-7(6H)-one hydrochloride (0.20 g) and (S)-3-((tert-butoxycarbonyl)(isopropyl)amino)-2-(4-chlorophenyl)-propionic acid (0.22 g) were dissolved in N,N-dimethylformamide (5 mL) at 20° C., 2-(7-benzotriazole oxide)-N,N,N′,N′-tetramethyluronium hexafluorophosphate (0.59 g) and 4-dimethylaminopyridine (0.48 g) were added, and the reaction solution reacted at 25° C. for 4 h. After the reaction was completed, water (20 mL) was added to the reaction solution, the mixture was extracted with ethyl acetate (10 mL×3), an obtained organic phase was washed with a saturated salt solution (10 mL×2), and the solution was evaporated under reduced pressure to remove the organic phase and separated by column chromatography (dichloromethane:methanol=50:1) to yield a yellow solid (0.18 g). LC-MS (ESI) m/z: 583 (M+H).

d) (R)-4-((1 S,6R)-5-((S)-2-(4-chlorophenyl)-3-(isopropylamino)propionyl)-2,5-diazabicyclo [4.1.0]heptan-2-yl)-5-met hyl-5,8-dihydropyrido[2,3-d]pyrimidin-7(6H)-one Tert-butyl
(2S)-2-(4-chlorophenyl)-3-(5-((R)-5-methyl-7-oxo-5,6,7,8-tetrahydropyrido[2,3-d]pyrimidin-4-yl)-2,5-diazabicyclo [4.1.0]heptan-2-yl)-3-oxopropyl)(isopropyl)carbamate (0.18 g) was dissolved in dichloromethane (2 mL) at 20° C., trifluoroacetic acid (0.86 mL) was added, and the reaction solution reacted for 3 h. After the reaction was completed, dichloromethane (10 mL) was added to the reaction solution, a 2 M sodium hydroxide solution was dropwise added at 0° C. to regulate the pH of the mixture to 12, the solution was separated, an obtained organic phase was washed with a saturated salt solution (5 mL), and the solution was dried with anhydrous sodium sulfate and evaporated under reduced pressure to remove the organic phase so as to yield a yellow solid (0.10 g). The yellow solid was resolved by preparative high-performance liquid chromatography to yield isomer 1 (3 mg) and isomer 2 (12 mg). Preparative high-performance liquid chromatography conditions: chromatographic column: Aglient 5 μm prep-C18 50×21.2 mm; mobile phase A: water (containing 0.1 vol % of ammonium water (25-28 wt %)); and mobile phase B: methanol. Gradient: time: 0-10 min, 60-70% (volume percentage) of B phase.

Isomer 1: RT₁=5.3 min; LC-MS (ESI) miz: 483 (M+H).

Isomer 2: RT=5.9 min; LC-MS (ESI) miz: 483 (M+H); ¹H NMR (400 MHz, CDCl₃) δ (ppm) 8.27 (d, J=7.6 Hz, 1H), 7.92 (s, 1H), 7.27-7.30 (m, 4H), 4.23-4.29 (m, 1H), 3.90-3.95 (m, 1H), 3.81-3.85 (m, 1H), 3.69-3.72 (m, 1H), 3.44-3.59 (m, 1H), 3.20-3.38 (m, 3H), 3.01-3.05 (m, 1H), 2.70-2.85 (m, 3H), 2.47-2.57 (m, 1H), 2.21-2.25 (m, 1H), 1.25-1.28 (m, 3H), 1.03-1.11 (m, 6H), 0.82-0.90 (m, 2H).

In the present application, configurations of the compounds of Example 1 were determined by single crystal diffraction, and it was determined that isomer 2 was compound 1 of the present application:

Preparation of a single crystal: isomer 2 (30.0 mg) and isopropanol (2.0 mL) were placed in a 5 mL screw flask and stirred for 5 min until the solid was fully dissolved. Oxalic acid dihydrate (3.9 mg) was weighed and placed in the above flask, a white solid was gradually precipitated in the flask, the reaction solution was stirred at the room temperature for 3 h, and a large amount of white solid was precipitated in the flask. Methanol (1.0 mL) was placed in the flask, the white solid gradually disappeared, and after becoming clear, the solution was stirred for 1 h. The solution was filtered with a 0.22 μm microfiltration membrane to a 3 mL screw flask, and the opening of the flask was covered with a plastic wrap. The plastic warp covering the opening of the flask was pierced by using a needle to form 8 small holes, the flask was placed at the room temperature for 7 d, and an oxalate single crystal of isomer 2 was obtained.
Single crystal diffraction experiment:
Single crystal X-ray diffractometer: BRUKER D8 VENTURE PHOTON II
Wavelength: Ga Kα(λ=1.34139 Å)
Test temperature: 190 K
Computer program for structural analysis: SHELXL-2018
Single crystal data: molecular formula: C₅₅H₇₂C₁₂N₁₂O₉; molecular weight: 1116.14; crystal system: hexagonal crystal system; space group: P61; cell parameters: a=25.8406(15) Å, b=25.8406(15) Å, c=45.916(3) Å, α=90°, β=90°, and γ=120°; unit cell volume: V=26552(4) Å³; the number of molecular formulas contained in the unit cell: Z=12; calculated density: D_{calc}=0.838 g/cm³; R_{W}(F₀²): 0.0730; R_{w}(F₀²): 0.2069; goodness of fit (S): 1.034; and Flack parameter: 0.066(9).

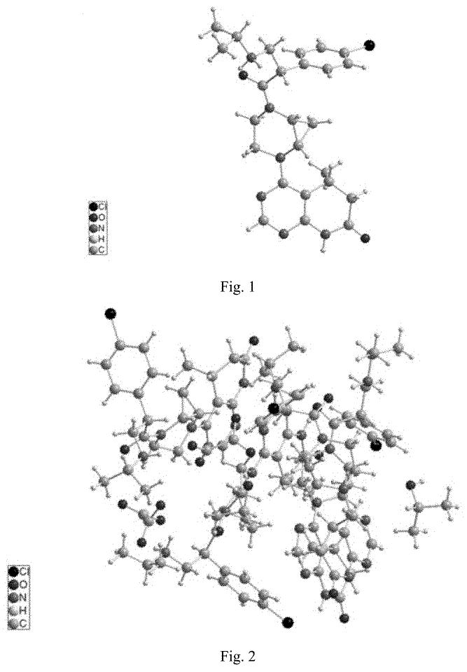

Structural description: single crystal X-ray diffraction and structural analysis show that the prepared single crystal is an oxalate isopropanol complex of isomer 2. Asymmetric structural unit of the crystal include four isomer 2 molecules, two oxalic acid molecules, and two isopropanol molecules, where isomer 2 and oxalic acid form an oxalate. The single molecule of isomer 2 is shown in FIG. 1, and the asymmetric structural unit of the oxalate single crystal are shown in FIG. 2. The structural formula is shown below:

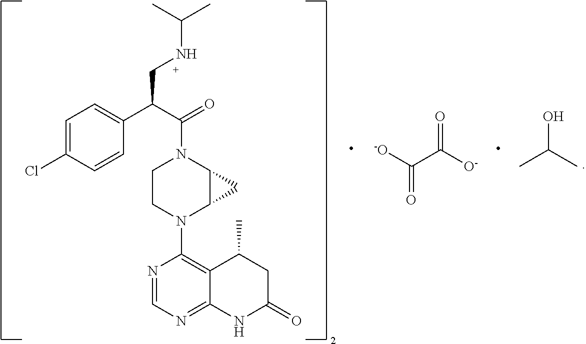

### Test Example 1 Test of AKT Kinase Inhibiting Activity

### 1. Materials and Reagents

Envision model plate reader (Molecular Devices)
White 384-well plate (Thermo, Art. No. #264706)
Main reagents included in an HTRF kinEASE TK kit (Cisbio, Art. No. #62TKOPEC)
TK-biotin substrate
Streptavidin-XL665
Europium-labeled tyrosine kinase substrate antibody
5× enzyme reaction buffer
SEB
HTRF assay buffer
AKT1 (Cama, Art. No. #01-101)
AKT2 (Cama, Art. No. #01-102)
AKT3 (Invitrogen, Art. No. #PV3185)
10 mM ATP (Invitrogen, Art. No. #PV3227)
1 M DTT (Sigma, Art. No. #D5545)
1 M MgCl₂ (Sigma, Art. No. #M8266)
Isomer 1 and isomer 2 of Example 1 of the present application
Positive control: GDC-0068

### 2. Experimental Procedure

### 2.1 Preparation of Reagents

TABLE 1
Concentrations of components of kinase reaction systems

| Reaction reagent | | AKT1 | | AKT2 | | AKT3 | |
|---|---|---|---|---|---|---|---|
| Concentration of enzyme | Final concentration at the | 0.6 | ng/well | 0.1 | ng/well | 0.3 | ng/well |
| Concentration of ATP | enzyme reaction step (10 μL) | 2 | μM | 20 | μM | 10 | nM |
| Concentration of TK-biotin substrate | | 2 | μM | 2 | μM | 2 | μM |
| Enzyme reaction time | | 50 | min | 50 | min | 50 | min |
| Concentration of streptavidin-XL665 | Final concentration in the | 125 | nM | 125 | nM | 125 | nM |

| | | | | |
|---|---|---|---|---|
| Concentration of europium-labeled | overall reaction (20 μL) | 1:100 | 1:100 | 1:100 |
| tyrosine kinase substrate antibody | | diluted | diluted | diluted |

1×kinase reaction buffer
A 1×kinase reaction buffer for 1 mL of kinase AKT1, AKT2 or AKT3 included 200 1 μL of 5×kinase reaction buffer,
5 μL, of 1 M MgCl₂, 1 μL, of 1 M DTT, and 794 μL of ultra-pure water.
5×TK-biotin substrate and ATP working solution
Specific concentrations of the TK-biotin substrate and ATP are shown in Table 1.
The substrate and ATP were respectively diluted with the 1×kinase reaction buffer to a concentration 5 times of the reaction concentration.
5×kinase working solution
The concentration for enzyme screening is shown in Table 1. A 5×enzyme working solution was prepared from the
1×kinase reaction buffer.
4×streptavidin-XL665 working solution
The concentration of streptavidin-XL665 in the reaction is shown in Table 1. A 4×streptavidin-XL665 working solution was prepared from the assay buffer.
4×europium-labeled tyrosine kinase substrate antibody working solution
The europium-labeled tyrosine kinase substrate antibody was 100-fold diluted with the assay reaction buffer to obtain a working solution.

### 2.2 Experimental Process

After all the reagents were prepared according to the above method, except for the enzyme, the reagents were equilibrated to the room temperature and loaded.

a) first, a compound stock solution (10 mM DMSO solution) was diluted with DMSO to obtain a 100 μM compound solution, the compound solution was diluted with the 1×kinase reaction buffer to obtain a 2.5 μM compound working solution (containing 2.5% DMSO). A 2.5% DMSO solution was prepared from the 1×kinase reaction buffer, and the 2.5 μM compound working solution was diluted 7 times with the 2.5% DMSO solution according to a 4-fold gradient to obtain compound working solutions at 8 concentrations (2500 nM, 625 nM, 156 nM, 39 nM, 9.8 nM, 2.4 nM, 0.6 nM, and 0.15 nM). Except for control wells, 4 μL of diluted compound working solution was placed in each reaction well, and 4 μL of previously prepared 2.5% DMSO/kinase buffer was placed in each control well.
b) 2 μL of previously prepared TK-biotin substrate solution (the concentration of the substrate for enzyme screening is shown in Table 1) was placed in each reaction well.
c) 2 μL of previously prepared enzyme solution (the concentration of the enzyme is shown in Table 1) was placed in each reaction well except for negative wells, and 2 μL of 1×kinase reaction buffer corresponding to the enzyme was placed in each negative well to make up the volume. The plate was sealed with a sealing film, and the reaction solution was mixed until uniform and incubated at the room temperature for 10 min to allow the compound to fully react with and bind to the enzyme.
d) 2 μL of ATP solution was placed in each reaction well to initiate a kinase reaction (the concentration of ATP for enzyme screening and reaction time are shown in Table 1).
e) 5 min before the kinase reaction was completed, an assay solution was prepared. Streptavidin-XL665 and a europium-labeled tyrosine kinase substrate antibody (1:100) assay solution (the concentration of the assay reagent is shown in Table 1) were prepared from the assay buffer in the kit.
f) After the kinase reaction was completed, 5 μL of diluted streptavidin-XL665 was placed in each reaction well and mixed with the reaction solution until uniform, and the diluted europium-labeled tyrosine kinase substrate antibody assay solution was immediately added.
g) The plate was sealed, the reaction solution was mixed until uniform and reacted at the room temperature for 1 h, and fluorescence signals were detected by using an ENVISION (Perkinelmer) instrument (320 nm stimulation, 665 nm, 615 nm emission). An inhibition rate in each well was calculated from all active wells and background signal wells, a mean value of repetitive wells was calculated, and the half inhibitory activity (IC50) of each compound to be tested was fitted by using the professional drawing analysis software PRISM 6.0.

TABLE 2
Experimental loading process

| | Kinase reaction | | |
|---|---|---|---|
| | system | Control group | |
| Enzyme reaction step (10 μL) | Sample group | Negative control | Positive control |
| Isomer 1 or isomer 2 of Example 1 | 4 μL | 4 μL of 2.5% | 4 μL of 2.5% |
| | | DMSO/kinase buffer | DMSO/kinase buffer |
| TK-biotin-labeled substrate | 2 μL | 2 μL | 2 μL |
| Kinase | 2 μL | 2 μL of kinase buffer | 2 μL |

Seal with a film, and incubate at the room temperature for 10 min

| | | | |
|---|---|---|---|
| ATP | 2 μL | 2 μL | 2 μL |

Seal with a film, and incubate at the room temperature for 50 min
Detection steps (10 μL)

| | | | |
|---|---|---|---|
| Streptavidin-XL665 | 5 μL | 5 μL | 5 μL |
| Europium-labeled tyrosine kinase | 5 μL | 5 μL | 5 μL |
| substrate antibody | | | |

Seal with a film, and incubate at the room temperature for 1 h
Detection light: 320 nm, emitted light: 665 nm, 615 nm

2.3 Data Analysis

ER=fluorescence value at 665 nm/fluorescence value at 615 nm

Inhibition rate=(ER_{positive control}−ERₛₐₘₚₗₑ)(ER_{positive control}−ER_{negative control})×100%

### 3. Experimental Results

Experimental results are shown in Table 3.

TABLE 3
AKT inhibiting activity

| | | AKT1 enzyme | AKT2 enzyme | AKT3 enzyme |
|---|---|---|---|---|
| | | activity | activity | activity |
| Compound | Chemical structure | IC₅₀ (nM) | IC₅₀ (nM) | IC₅₀ (nM) |
| Isomer 1 of Example 1 | 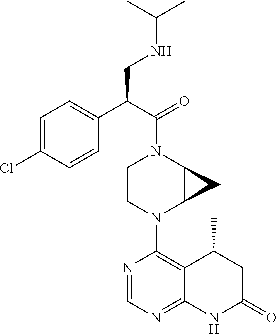 [SMILES: CC(C)NC[C@@H](C(=O)N1CCN(c2ncnc3c2[C@H](C)CC(=O)N3)[C@@H]2C[C@@H]21)c1ccc(Cl)cc1] | 62 | 542 | 13 |
| | Isomer 1 | | | |
| Isomer 2 of Example 1 (Compound 1) | 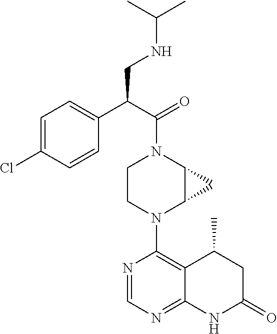 [SMILES: CC(C)NC[C@@H](C(=O)N1CCN(c2ncnc3c2[C@H](C)CC(=O)N3)[C@H]2C[C@H]21)c1ccc(Cl)cc1] | 0.35 | 6.3 | 0.09 |
| | Isomer 2 | | | |
| Positive control GDC-0068 | 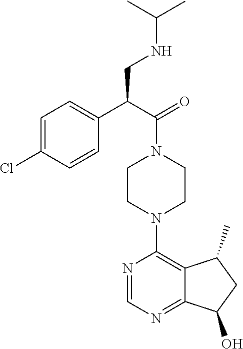 [SMILES: CC(C)NC[C@@H](C(=O)N1CCN(c2ncnc3c2[C@H](C)C[C@H]3O)CC1)c1ccc(Cl)cc1] | 3.2 | 1.7 | 2.5 |

### Example 2 Preparation of Crystal Form A

### (1) Method A: Preparation of Crystal Form a Using an Amorphous Fumarate of Compound 1 Preparation of an Amorphous Fumarate of Compound 1:

Compound 1 (25 mg) and isopropanol (1 mL) were placed in a 3 mL vial and magnetically stirred at the room temperature until the solid was fully dissolved. Solid fumaric acid (6.31 mg) was placed in the 3 mL vial, and the reaction solution was magnetically stirred at the room temperature for reaction. After the reaction solution was stirred for 18 h, n-heptane (2 mL) was placed in the 3 mL vial, and the reaction solution was stirred for 18 h. The reaction solution was subjected to suction filtration, and an obtained filter cake was dried in vacuum at 40° C. for 3 h to yield a white solid powdery amorphous fumarate of compound 1 that was characterized by ¹HNMR and XRPD.

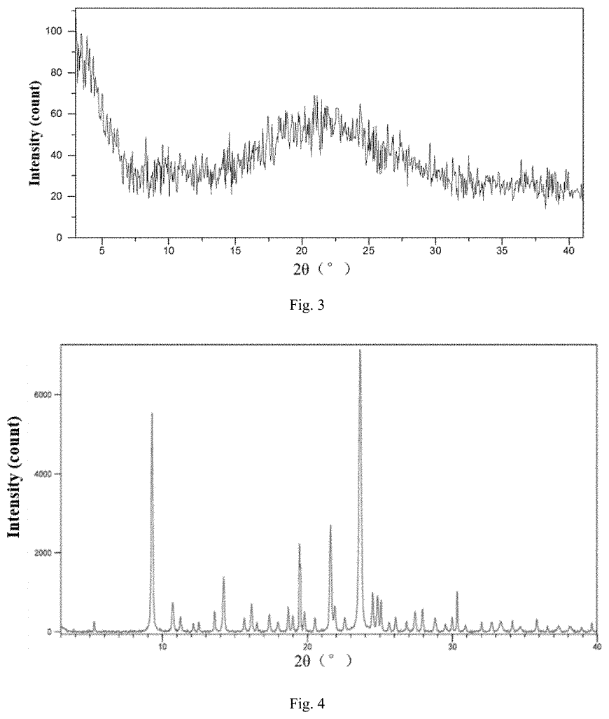

The XRPD pattern is shown in FIG. 3.

¹HNMR (400 MHz, DMSO-d₆): 10.49 (s, 1H), 8.20 (s, 1H), 7.34-7.48 (m, 4H), 6.52 (s, 2H), 4.37-4.76 (m, 1H), 3.88-4.18 (m, 1H), 3.70-3.81 (m, 2H), 3.34-3.54 (m, 2H), 3.03-3.21 (m, 4H), 2.90 (dd, J=11.6, 4.8 Hz, 1H), 2.76 (dd, J=16.4, 6.0 Hz, 1H), 2.22-2.30 (m, 1H), 1.04-1.32 (m, 8H), 0.85-0.93 (m, 4H), 0.08 (q, J=5.2 Hz, 1H).

Preparation of Crystal Form A

The amorphous fumarate of compound 1 (100 mg) and water (2 mL) were placed in a 3 mL vial and magnetically stirred at the room temperature until the solid was fully dissolved. After being stirred for 18 h, the solution was subjected to suction filtration, and an obtained wet filter cake was dried in vacuum at 40° C. for 5 h to yield white solid powdery crystal form A.

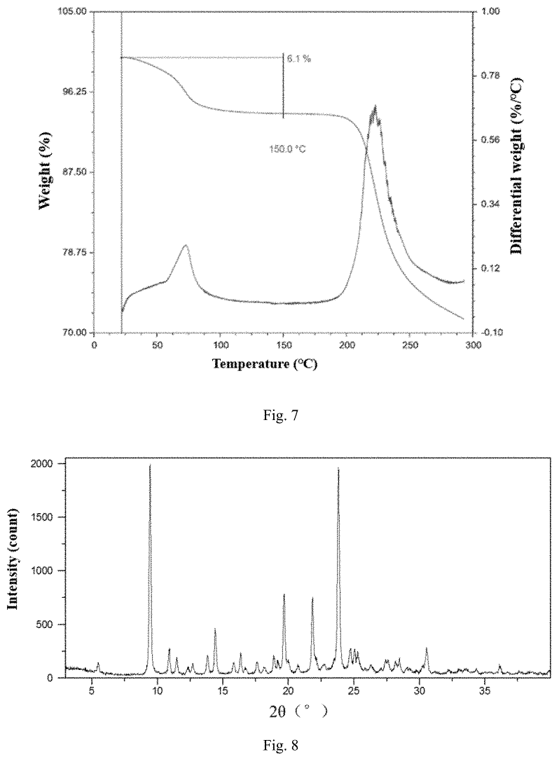

The TGA pattern is shown in FIG. 7, which shows that when crystal form A is heated to 150° C., the mass fraction of weight loss is about 6.1%.

The XRPD pattern is shown in FIG. 8.

(2) Method B: preparation of crystal form A by adding a seed crystal

Compound 1 (2 g) and acetone (10 mL) were placed in a 100 mL double-layer glass jacketed reactor and mechanically stirred at the room temperature. Solid fumaric acid (0.50 g) and ethanol/water (95:5 (v/v)) (7 mL) were placed in a 10 mL vial in sequence, heated to 60° C., and shaken until the solid was fully dissolved, and the temperature of the solution was maintained for later use. The above fumaric acid solution was placed in the reactor, and the reaction solution was cooled to the room temperature. A seed crystal (5.0 mg) of crystal form A of the fumarate was placed in the reactor and fully dissolved. After the reaction solution was cooled to 20° C., a seed crystal (5.0 mg) of crystal form A of the fumarate was placed in the reactor to induce crystallization, and the temperature of the reaction solution was maintained for 1.5 h. Then, the reaction solution was cooled to 10° C. and cured for 1.5 h. After being cured, the reaction solution was cooled to 2° C. After being cured, the reaction solution was heated to 20° C. and stirred at this temperature overnight. The reaction solution was subjected to suction filtration, and an obtained wet filter cake was dried in vacuum at 45° C. for 6 h to yield white needle-like crystal form A (0.7 g).

The mother solution was placed back into the reactor, n-heptane (20 mL) was added, and the reaction solution was stirred and cured at the room temperature. The reaction solution was subjected to suction filtration, and an obtained wet filter cake was dried in vacuum at 45° C. for 6 h to yield white solid powdery crystal form A (1.1 g). The crystal form was respectively characterized by ¹HNMR, XRPD, DSC, TGA, FT-IR, and FT-Raman.

¹HNMR (400 MHz, DMSO-d₆): 10.49 (s, 1H), 8.20 (s, 1H), 7.34-7.48 (m, 4H), 6.52 (s, 2H), 4.40-4.77 (m, 1H), 3.88-4.18 (m, 1H), 3.69-3.80 (m, 2H), 3.35-3.54 (m, 2H), 3.08-3.21 (m, 4H), 2.91 (dd, J=11.6, 4.4 Hz, 1H), 2.76 (dd, J=16.0, 6.0 Hz, 1H), 2.22-2.30 (m, 1H), 1.06-1.30 (m, 8H), 0.76-0.99 (m, 4H), 0.08 (q, J=4.8 Hz, 1H). XRPD characteristic peaks of crystal form A are shown in Table 4 and FIG. 4.

TABLE 4
XRPD characteristic peaks of crystal form A

| 2θ (°) | I/I₀ (%) |
|---|---|
| 5.29 | 3.6 |
| 9.28 | 77.4 |
| 10.72 | 10.5 |
| 11.24 | 5.2 |
| 12.13 | 2.8 |
| 12.51 | 3.3 |
| 13.60 | 7.1 |
| 14.22 | 19.2 |
| 15.64 | 4.8 |
| 16.14 | 9.8 |
| 16.52 | 3.3 |
| 17.38 | 6.2 |
| 17.99 | 3.3 |
| 18.68 | 8.7 |
| 19.00 | 5.7 |
| 19.45 | 31.5 |
| 19.80 | 7.0 |
| 20.53 | 4.8 |
| 21.60 | 37.6 |
| 21.89 | 9.0 |
| 22.58 | 5.1 |
| 23.63 | 100.0 |
| 24.50 | 13.7 |
| 24.83 | 12.6 |
| 25.08 | 11.1 |
| 25.66 | 3.0 |
| 26.09 | 5.2 |
| 26.84 | 3.6 |
| 27.43 | 7.1 |
| 27.94 | 7.9 |
| 28.81 | 4.9 |
| 29.52 | 2.6 |
| 29.98 | 5.1 |
| 30.33 | 14.2 |
| 30.92 | 2.5 |
| 32.03 | 3.3 |
| 32.80 | 1.5 |
| 33.34 | 3.6 |
| 34.14 | 3.8 |
| 34.72 | 1.6 |
| 35.83 | 4.3 |
| 36.55 | 2.0 |
| 37.35 | 2.0 |
| 38.11 | 2.0 |
| 38.93 | 1.5 |

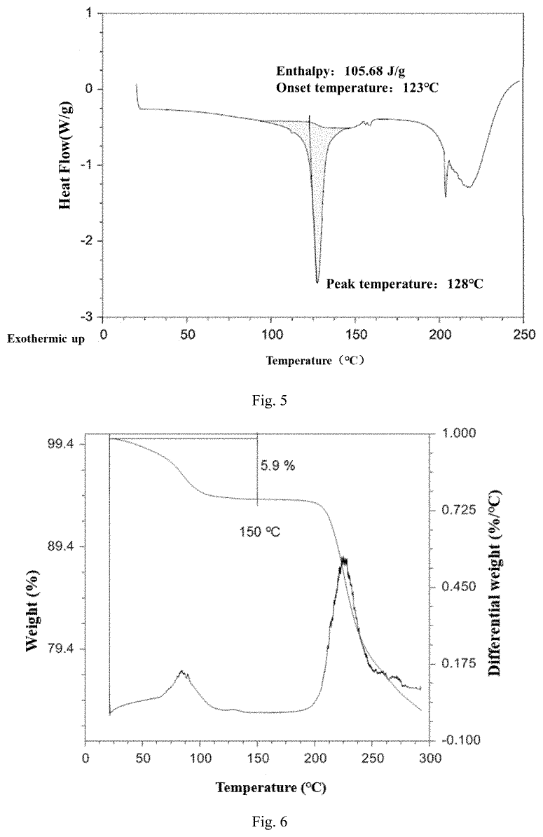

The DSC pattern of crystal form A is shown in FIG. 5, which shows that the onset temperature and peak temperature of endothermic peak are respectively 123° C. and 128° C.

An infrared spectrum of crystal form A that is obtained by attenuated total reflectance Fourier transform infrared spectroscopy (FT-IR) has the following absorption bands expressed in reciprocals of wavelengths (cm⁻¹): 3451±2, 2981±2, 2953±2, 2882±2, 2824±2, 2477±2, 1698±2, 1631±2, 1596±2, 1544±2, 1490±2, 1465±2, 1441±2, 1390±2, 1362±2, 1320±2, 1302±2, 1283±2, 1254±2, 1197±2, 1135±2, 1091±2, 1058±2, 1014±2, 983±2, 929±2, 894±2, 867±2, 834±2, 802±2, 784±2, 761±2, 739±2, 718±2, 663±2, 647±2, 640±2, 584±2, 560±2, and 497±2.

A Raman spectrum of crystal form A that is obtained by Fourier transform Raman spectroscopy (FT-Raman) has the following absorption bands expressed in reciprocals of wavelengths (cm-1): 1699±2, 1664±2, 1602±2, 1340±2, 867±2, 829±2, 809±2, 747±2, and 669±2.

The TGA pattern is shown in FIG. 6, which shows that when crystal form A is heated to 150° C., the mass fraction of weight loss is about 5.9%.

It can be seen that the crystal forms of the fumarates of compound 1 that are prepared by method A and method B are identical.

### Example 3 Preparation of Crystal Form a by Adding a Seed Crystal

Compound 1 (5 g) and acetone (25 mL) were placed in a 100 mL double-layer glass jacketed reactor in sequence, heated to 45° C., and mechanically stirred until the solid was fully dissolved. Solid fumaric acid (1.26 g) and an ethanol/water binary solvent (95:5 (v/v)) (17.5 mL) were placed in a 20 mL vial in sequence, heated to 60° C., and shaken until the solid was fully dissolved, and the temperature of the solution was maintained for later use. The above fumaric acid solution was placed in the reactor, and the reaction solution was cooled to 45° C. N-heptane (12.5 mL) and a seed crystal (5 mg) of crystal form A were placed in the reactor in sequence, and the reaction solution was stirred for 30 min. N-heptane (10.0 mL) and a seed crystal (5 mg) of crystal form A of the fumarate were placed in the reactor in sequence to induce crystallization, and the temperature of the reaction solution was maintained while the reaction solution was cured for 1 h. N-heptane (27.5 mL) was placed in the reactor, and the reaction solution was naturally cooled to the room temperature and stirred overnight. The reaction solution was subjected to suction filtration, and an obtained wet filter cake was dried in vacuum at 45° C. for 4 h to yield white solid powdery crystal form A (2.8 g).

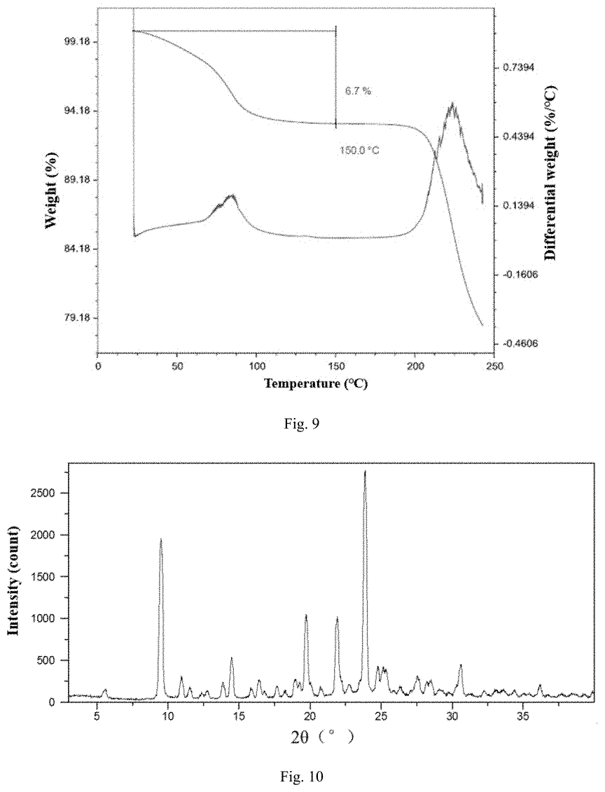

The TGA pattern is shown in FIG. 9, which shows that when the crystal form is heated to 150° C., the mass fraction of weight loss is about 6.7%.

The XRPD pattern is shown in FIG. 10.

### Example 4 Stability of Crystal Form A

The solid stability of crystal form A prepared in Example 3 was tested under the following preservation conditions.
a. Hot and humid conditions: temperature: 40° C., relative humidity: 75%, crystal form A was exposed to air for 20 d
b. High temperature conditions: temperature: 60° C., crystal form A was exposed to air for 20 d

The chemical purity of crystal form A was measured by HPLC.

Chromatographic column: ACE Excel 5 Super C18 (4.6×150 mm, 5 μm)

Detection wavelength: 230 nm, column temperature: 30° C., flow rate: 1.0 mL/min

Mobile phase: diammonium hydrogen phosphate (1.32 g) was weighed and dissolved in water (1000 mL), the solution was adjusted with phosphoric acid until the pH of the solution was 7.2, filtered, and used as A phase; and acetonitrile was used as B phase.

Gradient conditions:

| Time (min) | A phase (%) | B phase (%) |
|---|---|---|
| 0 | 90 | 10 |
| 5 | 90 | 10 |
| 50 | 15 | 85 |
| 55 | 15 | 85 |
| 55.5 | 90 | 10 |
| 60 | 90 | 10 |

Test results are shown blow:

Purity (%)

| Placement | Before placement | After placement |
|---|---|---|
| conditions | (day 0) | (day 20) |
| a | 99.84 | 99.89 |
| b | 99.84 | 99.88 |

In the present application, as demonstrated by Test Example 1 above, compound 1 of the present application has an inhibiting effect on the AKT kinase activity, and correspondingly, the crystal form of the fumarate hydrate of compound 1 of the present application also has an inhibiting effect on the AKT kinase activity. Therefore, the crystal form of the fumarate hydrate of compound 1 of the present application, and a crystal form composition and pharmaceutical composition including the crystal form can be used for preventing and/or treating an AKT protein kinase-mediated disease or disease state, and further can be used for preparing a medicament for preventing and/or treating an AKT protein kinase-mediated disease or disease state. Much further, the crystal form of the fumarate hydrate of compound 1 of the present application has higher stability, the physical and chemical properties of compound 1 are improved, and optimizes the bioavailability, so it is more favorable for production and application.

The above are preferred embodiments of the present application only, but are not intended to limit the present application. Any modification, equivalent replacement, and improvement made within the spirit and principle of the present application shall fall within the protection scope of the present application.

What is claimed is:

1. A crystal form of a fumarate hydrate having the following structure, wherein the crystal form is a crystal form A, 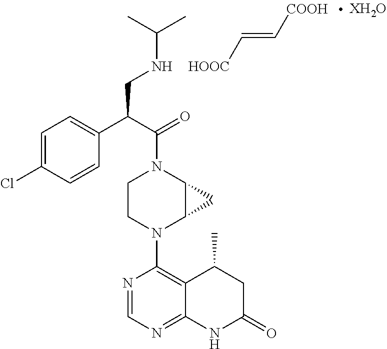
wherein X is 2.0-3.0, and
an X-ray powder diffraction pattern expressed in 2θ angles using Cu-Ka radiation has characteristic peaks at 2θ values of 9.28°±0.2° and 3.63°±0.2°.

2. The crystal form according to claim 1, wherein the X-ray powder diffraction pattern expressed in 2θ angles has characteristic peaks at 2θ values of 9.28°±0.2°, 19.45°±0.2°, 21.60°±0.2°, and 23.63°±0.2°.

3. The crystal form according to claim 1, wherein the X-ray powder diffraction pattern expressed in 2θ angles has characteristic peaks at 2θ values of 9.28°±0.2°, 14.22°±0.2°, 19.45°±0.2°, 21.60°±0.2°, and 23.63°±0.2°.

4. The crystal form according to claim 1, wherein the X-ray powder diffraction pattern expressed in 2θ angles has characteristic peaks at 2θ values of 9.28°±0.2°, 10.72°±0.2°, 14.22°±0.2°, 19.45°±0.2°, 21.60°±0.2°, 23.63°±0.2°, 24.50°±0.2°, 24.83°±0.2°, 25.08°±0.2°, and 30.33°±0.2°.

5. The crystal form according to claim 1, wherein the X-ray powder diffraction pattern expressed in 2θ angles has characteristic peaks at 2θ values of 5.29°±0.2°, 9.28°±0.2°, 10.72°±0.2°, 11.24°±0.2°, 12.13°±0.2°, 12.51°±0.2°, 13.60°±0.2°, 14.22°±0.2°, 15.64±0.2°, 16.14°±0.2°, 16.52°±0.2°, 17.38°±0.2°, 17.99°±0.2°, 18.68°±0.2°, 19.00°±0.2°, 19.45°±0.2°, 19.80°±0.2°, 20.53°±0.2°, 21.60°±0.2°, 21.89°±0.2°, 22.58°±0.2°, 23.63°±0.2°, 24.50°±0.2°, 24.83°±0.2°, 25.08°±0.2°, 25.66°±0.2°, 26.09°±0.2°, 26.84°±0.2°, 27.43°±0.2°, 27.94°±0.2°, 28.81°±0.2°, 29.52°±0.2°, 29.98°±0.2°, 30.33°±0.2°, 30.92°±0.2°, 32.03°±0.2°, 32.80°±0.2°, 33.34°±0.2°, 34.14°±0.2°, 34.72°±0.2°, 35.83°±0.2°, 36.55°±0.2°, 37.35°±0.2°, 38.11°±0.2°, and 38.93°±0.2°.

6. The crystal form according to claim 1, wherein the X-ray powder diffraction pattern expressed in 2θ angles is shown as FIG. 4, or shown as FIG. 8, or shown as FIG. 10.

7. The crystal form according to claim 1, wherein a thermogram of the crystal form A obtained by differential scanning calorimetry (DSC) has an endothermic peak at an onset temperature of 118-128° C.; preferably has an endothermic peak at an onset temperature of 120-125° C., and more preferably has an endothermic peak at an onset temperature of 123° C.; and more preferably, the DSC pattern is shown as FIG. 5.

8. The crystal form according to claim 1, wherein a spectrum of the crystal form A obtained by attenuated total reflectance Fourier transform infrared spectroscopy has the following absorption bands expressed in reciprocals of wavelengths (cm⁻¹): 3451±2, 2981±2, 2953±2, 2882±2, 2824±2, 2477±2, 1698±2, 1631±2, 1596±2, 1544±2, 1490±2, 1465±2, 1441±2, 1390±2, 1362±2, 1320±2, 1302±2, 1283±2, 1254±2, 1197±2, 1135±2, 1091±2, 1058±2, 1014±2, 983±2, 929±2, 894±2, 867±2, 834±2, 802±2, 784±2, 761±2, 739±2, 718±2, 663±2, 647±2, 640±2, 584±2, 560±2, and 497±2.

9. The crystal form according to claim 1, wherein a spectrum of the crystal form A obtained by Fourier transform Raman spectroscopy has the following absorption bands expressed in reciprocals of wavelengths (cm⁻¹): 1699±2, 1664±2, 1602±2, 1340±2, 867±2, 829±2, 809±2, 747±2, and 669±2.

10. The crystal form according to claim 1, wherein the TGA pattern is shown as FIG. 6, or shown as FIG. 7, or shown as FIG. 9.

11. A preparation method of the crystal form according to claim 1, comprising a step of adding a seed crystal of the crystal form A during a salification reaction of compound 1 with fumaric acid; or dissolving an amorphous fumarate of the compound 1 in water, and performing a suction filtration and a vacuum drying, wherein the compound 1 has the following structure:

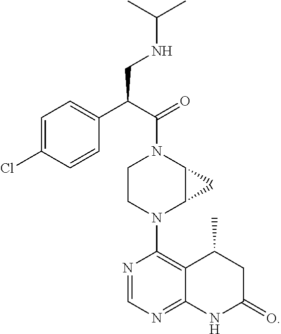 [SMILES: CC(C)NC[C@@H](C(=O)N1CCN(c2ncnc3c2[C@H](C)CC(=O)N3)[C@H]2C[C@H]21)c1ccc(Cl)cc1]

12. A crystal form composition, comprising the crystal form according to claim 1, wherein the weight of the crystal form accounts for more than 50% of the weight of the crystal form composition.

13. A pharmaceutical composition, comprising the crystal form according to claim 1 or a crystal form composition thereof, wherein the crystal form composition comprises the crystal form, and the weight of the crystal form accounts for more than 50% of the weight of the crystal form composition.

14. The crystal form according to claim 1, or a crystal form composition thereof, or a pharmaceutical composition thereof for use as a medicament, wherein the crystal form composition comprises the crystal form, and the weight of the crystal form accounts for more than 50% of the weight of the crystal form composition; the pharmaceutical composition comprises the crystal form or the crystal form composition.

15. A method of a use of the crystal form according to claim 1, or a crystal form composition thereof, or a pharmaceutical composition thereof in a prevention and/or a treatment of an AKT protein kinase-mediated disease or disease state, wherein the crystal form composition comprises the crystal form, and the weight of the crystal form accounts for more than 50% of the weight of the crystal form composition; the pharmaceutical composition comprises the crystal form or the crystal form composition.

16. A method of a use of the crystal form according to claim 1, or a crystal form composition thereof, or a pharmaceutical composition thereof in a preparation of a medicament for preventing and/or treating an AKT protein kinase-mediated disease or disease state, wherein the crystal form composition comprises the crystal form, and the weight of the crystal form accounts for more than 50% of the weight of the crystal form composition; the pharmaceutical composition comprises the crystal form or the crystal form composition.

17. The method of the use according to claim 15, wherein the AKT protein kinase-mediated disease or disease state is a cancer, preferably a breast cancer, a prostate cancer, or an ovarian cancer, and more preferably the prostate cancer.

18. A method for preventing and/or treating an AKT protein kinase-mediated disease or disease state, comprising a step of administering the crystal form according to claim 1, or a crystal form composition thereof, or a pharmaceutical composition thereof to a subject in need, wherein the crystal form composition comprises the crystal form, and the weight of the crystal form accounts for more than 50% of the weight of the crystal form composition; the pharmaceutical composition comprises the crystal form or the crystal form composition.

19. The method according to claim 18, wherein the AKT protein kinase-mediated disease or disease state is a cancer, preferably a breast cancer, a prostate cancer, or an ovarian cancer, and more preferably the prostate cancer.

20. The method of the use according to claim 16, wherein the AKT protein kinase-mediated disease or disease state is a cancer, preferably a breast cancer, a prostate cancer, or an ovarian cancer, and more preferably the prostate cancer.
